(19) United States
(12) Patent Application Publication
(10) Pub. No.: US 2021/0186131 A1
(43) Pub. Date: Jun. 24, 2021

(54) Enhanced Grip Heat Resistant Disposable Dental Medical Gloves

(71) Applicant: Donata Lee DuPree, Kingman, AZ (US)

(72) Inventor: Donata Lee DuPree, Kingman, AZ (US)

(21) Appl. No.: 16/919,434

(22) Filed: Jul. 28, 2020

(51) Int. Cl. A41D 19/015; A61B 42/10

(52) U.S. Cl. CPC A41D 19/01558; A61B 42/10; A41D 19/01529

(57) ABSTRACT

Disposable (or multi-use) gloves are made of latex, nitrile, or any other common material seen in current medical/dental gloves, with the addition of a thicker neoprene, polyurethane, polysuede, or suede-like material overlaid, or embedded, at the tips to allow for protection from heat or cold and enhanced grip for small (possibly wet) items such as crowns.

Gloves can have multiple applications: As described above, for dental hygienists, nurses, doctors, laboratory personnel, jewelers, watch makers, veterinarians, vet techs, do-it-yourselfers/employees working on automobiles/computers/soldering, crafters, anyone working on projects requiring enhanced finger grip as well as fingertip protection from extreme heat or cold.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

### (1) Field of the Invention

Jul. 2, 2019: I was at Dr. Kelley Bradley's office (DDS) getting a gold crown placed. Dr. Bradley was fitting and putting the final touches on the crown by grinding/polishing it. I asked her how many crowns have shot across the room during that process, she said more than you′d think. She said actually the worst problem was that she′d burn her fingertips as the crowns heat up during the grinding/polishing stage. I said someone should invent gloves to protect dentist's fingertips while working on crowns. Jul. 3, 2019: I told Rene Delzin about yesterday's conversation and decided to research this idea to see if someone had already invented disposable medical gloves with protection at the fingertips of the thumb, index, and 2^{nd} fingers. I envision typical disposable gloves with squares of something like suede glued/embedded on the palmar surface of the finger tips. Research did not turn up anything like this, only disposable medical gloves that have extra ‘grip’ ridges, or thickness, at the fingertips. Next, I researched the US patent process and the following is based on their questions on the uspto.gov site: The purpose of the gloves is as above and is a utilitarian device. This is a product, not a process.

### (2) Description of Related Art Including Information Disclosed Under 37 CFR 1.97 and 1.98

The gripping surfaces of the thumb, first, and second fingertips of both hands have areas of overlaid, or embedded, material to allow for protection from heat or cold and affording an enhanced grip for small (possibly wet) items such as dental crowns while the item is being ground or polished.

Prior art relates to my invention as outlined in the following search:

Search at patent website for A41D Outerwear; Protective Garments; Accessories shows CPC A41D 19/01564 {using strips of material} and CPC A41D 19/01547 {with grip improving means} and CPC A41D 19/01558 {using a layer of grip improving material (A41D 19/01552 takes precedence—this is using hooks and loops fastening system)}.

Search of issued patents using above CPC classifications returned:

A non-slip material including a basic cloth layer, an adhesive layer on a back surface of the basic cloth layer, a detachable paper sheet attached over the adhesive layer until use, a net attached to the opposite surface of the basic cloth layer, and a resin coating layer formed on the net so as to cover the surface of the net and the basic cloth layer. A method of making a non-slip material includes forming of an adhesive layer on the back of a basic cloth layer, attaching a detachable paper sheet to the adhesive layer, bonding a net on the opposite surface of the basic cloth layer, and forming a resin coating layer on the surface of the net and the basic cloth layer to which the net has been bonded. Filed Mar. 27, 1990

Textured gloves: Provided among other things is a glove formed by latex dipping having one or more textured finger tips wherein: the texture at the finger tips results from forming the glove on a former having the following properties: TABLE-US-00001 (micrometer) Ra Av. roughness about 7-14 Rsm Mean width of roughness about 500-720 Rpc Peak count about 15-20 the glove being non-chlorinated, wherein the glove is a (i) single dip nitrile glove or (ii) a dipped glove with a textured layer formed of polymer latex, and comprising a multivalent metal ion salt of an organic acid with log P of about 4 to about 15. U.S. Pat. No. 10,058,137 filed Oct. 12, 2015.

Layered structure with barrier layer, apparel piece with such a layered structure and production of such a layered structure: A layered structure that includes at least a first layer and a second layer is provided. The second layer includes at least one barrier layer. A bonding substance is partially disposed between the barrier layer and the first layer to create a bond between the barrier layer and the first layer. The bonding substance contains a first component that has, at 25.degree. C., a shear modulus of not more than 3.times.10.sup.5 Pa in accordance with the Dahlquist criterion. The bonding substance may also contain a second component having adhesive characteristics. The first component also has a low viscosity such that a force (F) between the first and second layers which acts on at least one of the layers in a force action area is distributed by a plastic flow onto an area which is larger than the force action area. Patent #10,005,258 filed Jun. 26, 2018.

Glove providing grip and dexterity: A glove that provides finger dexterity and grip while keeping the wearer's hands warm. The glove includes an exposed tight inner layer on the palm side of the fingertips to provide a good gripping surface with the glove on. The only exposed portion of the tight layer may be on the side of the finger where there are fingerprints—the palm side. The back side of the glove, where the fingernails and knuckles are, may be covered by a normal glove. The outer layer is cut out on the palm side and attached to the tight layer along the edge of the palm of the hand. The tight layer of the glove could either only be on the fingertips, or could be tight on the entire palm side of the glove to expose the whole palm to the gripping surface. This may require the tight inner lining around the whole hand. U.S. Pat. No. 9,642,406 filed May 9, 2017.

Utility glove: A utility glove formed of a glove blank of a fabric material in the shape of at least a portion of a hand having one or more areas of elastomeric material injection molded thereon to form one or more three dimensional molded portions, the three dimensional molded portions being the fabric material with the elastomeric material bonded thereto, wherein one or more three dimensional molded portions leave at least one area of fabric material of the glove blank without elastomeric material bonded to the fabric. The fabric material of the glove blank can be made from one or more fabrics, a treated fabric, a coated fabric or combinations thereof, or the fabric may have a portion with a coating on an area without elastomeric material. U.S. Pat. No. 9,555,567 filed Jan. 31, 2017.

I did not locate any similar products that are currently patented.

### BRIEF SUMMARY OF THE INVENTION

The gloves are made of latex, nitrile, or any other common material seen in current disposable medical/dental gloves, with the addition of a thicker neoprene, polyurethane, polysuede, or suede-like material overlaid, or embedded, at the tips to allow for protection from heat or cold and enhanced grip for small (possibly wet) items such as crowns.

### BRIEF DESCRIPTION OF THE SEVERAL VIEWS OF THE DRAWING(S)

FIG. 1 shows Element 1, disposable latex, nitrile, or similar material medical- or dental-type five digit gloves constructed and arranged to fit the hands and wrists of the user and comprising of enhanced grip heat and cold resistant pads disposed on the fingertips of the first and second and thumb digits of the gloves.

Element 2 shows the fingers of said gloves.

Element 3 illustrates the addition of a thicker neoprene, polyurethane, polysuede, or suede-like material overlaid, or embedded, at the fingertips to allow for protection from heat or cold as well as offer an enhanced grip for small (possibly wet) items.

### DETAILED DESCRIPTION OF THE INVENTION

The gloves are made of latex, nitrile, or any other common material seen in current disposable medical/dental gloves, with the addition of a thicker neoprene, polyurethane, polysuede, or suede-like material overlaid, or embedded, at the tips to allow for protection from heat or cold and enhanced grip for small (possibly wet) items such as crowns.

The keywords/technical terms that describe the nature of the gloves are as above and can also be latex exam gloves, disposable gloves, industrial gloves, medical gloves, nitrile gloves, vinyl gloves, protective gloves, surgical gloves, powder-free medical gloves, neoprene exam gloves, textured exam gloves, powder-free exam gloves, and hand gloves.

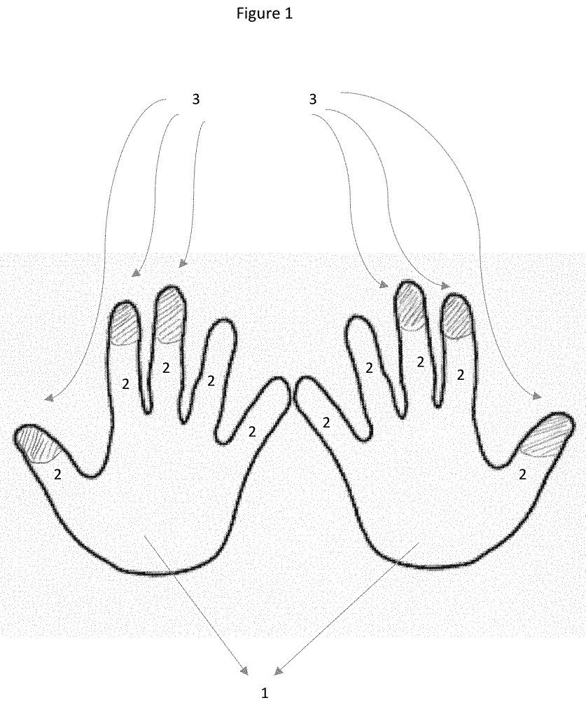

As seen in FIG. 1, these gloves are further broken down into: Element 1, which are disposable latex, nitrile, or similar material medical- or dental-type five digit gloves constructed and arranged to fit the hands and wrists of the user and comprising of enhanced grip heat and cold resistant pads disposed on the fingertips of the first and second and thumb digits of the gloves.

Element 2 shows the fingers of said gloves.

Element 3 illustrates the addition of a thicker neoprene, polyurethane, polysuede, or suede-like material overlaid, or embedded, at the fingertips to allow for protection from heat or cold as well as offer an enhanced grip for small (possibly wet) items.

Search at patent website for A41D Outerwear; Protective Garments; Accessories shows CPC A41D 19/01564 {using strips of material} and CPC A41D 19/01547 {with grip improving means} and CPC A41D 19/01558 {using a layer of grip improving material (A41D 19/01552 takes precedence—this is using hooks and loops fastening system)}.

Search of issued patents using above CPC classifications returned:

A non-slip material including a basic cloth layer, an adhesive layer on a back surface of the basic cloth layer, a detachable paper sheet attached over the adhesive layer until use, a net attached to the opposite surface of the basic cloth layer, and a resin coating layer formed on the net so as to cover the surface of the net and the basic cloth layer. A method of making a non-slip material includes forming of an adhesive layer on the back of a basic cloth layer, attaching a detachable paper sheet to the adhesive layer, bonding a net on the opposite surface of the basic cloth layer, and forming a resin coating layer on the surface of the net and the basic cloth layer to which the net has been bonded. Filed Mar. 27, 1990

Textured gloves: Provided among other things is a glove formed by latex dipping having one or more textured finger tips wherein: the texture at the finger tips results from forming the glove on a former having the following properties: TABLE-US-00001 (micrometer) Ra Av. roughness about 7-14 Rsm Mean width of roughness about 500-720 Rpc Peak count about 15-20 the glove being non-chlorinated, wherein the glove is a (i) single dip nitrile glove or (ii) a dipped glove with a textured layer formed of polymer latex, and comprising a multivalent metal ion salt of an organic acid with log P of about 4 to about 15. U.S. Pat. No. 10,058,137 filed Oct. 12, 2015.

Layered structure with barrier layer, apparel piece with such a layered structure and production of such a layered structure: A layered structure that includes at least a first layer and a second layer is provided. The second layer includes at least one barrier layer. A bonding substance is partially disposed between the barrier layer and the first layer to create a bond between the barrier layer and the first layer. The bonding substance contains a first component that has, at 25.degree. C., a shear modulus of not more than 3.times.10.sup.5 Pa in accordance with the Dahlquist criterion. The bonding substance may also contain a second component having adhesive characteristics. The first component also has a low viscosity such that a force (F) between the first and second layers which acts on at least one of the layers in a force action area is distributed by a plastic flow onto an area which is larger than the force action area. U.S. Pat. No. 10,005,258 filed Jun. 26, 2018.

Glove providing grip and dexterity: A glove that provides finger dexterity and grip while keeping the wearer's hands warm. The glove includes an exposed tight inner layer on the palm side of the fingertips to provide a good gripping surface with the glove on. The only exposed portion of the tight layer may be on the side of the finger where there are fingerprints—the palm side. The back side of the glove, where the fingernails and knuckles are, may be covered by a normal glove. The outer layer is cut out on the palm side and attached to the tight layer along the edge of the palm of the hand. The tight layer of the glove could either only be on the fingertips, or could be tight on the entire palm side of the glove to expose the whole palm to the gripping surface. This may require the tight inner lining around the whole hand. U.S. Pat. No. 9,642,406 filed May 9, 2017.

Utility glove: A utility glove formed of a glove blank of a fabric material in the shape of at least a portion of a hand having one or more areas of elastomeric material injection molded thereon to form one or more three dimensional molded portions, the three dimensional molded portions being the fabric material with the elastomeric material bonded thereto, wherein one or more three dimensional molded portions leave at least one area of fabric material of the glove blank without elastomeric material bonded to the fabric. The fabric material of the glove blank can be made from one or more fabrics, a treated fabric, a coated fabric or combinations thereof, or the fabric may have a portion with a coating on an area without elastomeric material. U.S. Pat. No. 9,555,567 filed Jan. 31, 2017.

I did not locate any similar products that are currently patented.

Patent application filed for Disposable exam gloves with enhanced, and heat/cold-resistant, grip.

Invented by Donata Lee DuPree, RN.

Not created under a government contract.

Gloves can have multiple applications: As described above, for dental hygienists, nurses, doctors, laboratory personnel, jewelers, watch makers, veterinarians, vet techs, do-it-yourselfers/employees working on automobiles/computers/soldering, crafters, anyone working on projects requiring enhanced finger grip as well as fingertip protection from extreme heat or cold.

1. A disposable latex, nitrile, or similar material medical- or dental-type five digit glove constructed and arranged to fit the hand of the user comprising enhanced grip heat and cold resistant pads disposed on the fingertips of the first and second and thumb digits of said glove.
